(19) United States
(12) Patent Application Publication
(10) Pub. No.: US 2026/0100287 A1
(43) Pub. Date: Apr. 9, 2026

(54) AI-POWERED ULTRASOUND SCAN REVIEW GENERATOR

(71) Applicant: Global Ultrasound Institute, Inc., Walnut Creek, CA (US)

(72) Inventors: Douglas Williams, Harvard, MA (US); Svetlana Zacharchenko, Fort Lee, NJ (US)

(73) Assignee: Global Ultrasound Institute, Inc., Walnut Creek, CA (US)

(21) Appl. No.: 18/907,254

(22) Filed: Oct. 4, 2024

(51) Int. Cl. G16H 70/60; G16H 30/40

(52) U.S. Cl. CPC G16H 70/60; G16H 30/40

(57) ABSTRACT

A system and method are disclosed which pertain to generating feedback on ultrasound scans. The system includes an artificial intelligence (AI) model, trained on a diverse dataset of ultrasound scans and associated findings, which processes scan data to generate a set of assessment facts. A user interface, driven by per-exam configuration, allows the reviewer to select appropriate items based on findings accuracy, scan completeness, and other teaching points. The AI model synthesizes the assessment facts and instructions to generate a written review of the ultrasound scan. The system can provide feedback in multiple languages and is adaptable to various types of ultrasound scans and healthcare settings.

### FIELD OF THE INVENTION

The present technology relates generally to medical imaging technology, and more particularly, to an artificial intelligence system for generating feedback on ultrasound scans.

### INTRODUCTION

Ultrasound imaging, also known as sonography, is a widely used diagnostic tool in healthcare. It leverages high-frequency sound waves to produce images of structures within the body. Ultrasound technology is non-invasive and offers real-time imaging, making it a valuable tool for various medical applications, including the examination of internal organs, blood vessels, and tissues, as well as monitoring the development of fetuses during pregnancy.

The quality and usefulness of ultrasound imaging largely depend on the skill and experience of the healthcare practitioner performing the scan. Proper manipulation of the ultrasound probe, appropriate selection of ultrasound settings, and accurate interpretation of the resulting images are all integral to the successful application of ultrasound in clinical practice.

Training healthcare practitioners to proficiently perform and interpret ultrasound scans typically involves a substantial amount of hands-on practice, supplemented by expert feedback. This feedback is often provided by experienced sonographers or radiologists who review the ultrasound scans performed by the trainee and provide guidance on areas of improvement. This feedback process is a cornerstone of competency development in ultrasound practice.

However, the process of providing expert feedback on ultrasound scans can be time-consuming and labor-intensive. It requires the expert reviewer to meticulously examine each scan, assess the accuracy and completeness of the images, and formulate constructive feedback that can guide the trainee's learning and improvement. Given the increasing demand for ultrasound skills across various healthcare specialties and the limited availability of expert reviewers, this traditional feedback process poses considerable challenges to growth and development within ultrasound imaging.

Moreover, the feedback process is not limited to training scenarios. Even in routine clinical practice, the review of ultrasound scans by a second practitioner can be a valuable quality assurance measure. However, the same challenges of time and resource constraints apply, making it difficult to implement consistent scan reviews in many healthcare settings.

In addition to these challenges, the feedback process also involves a degree of subjectivity, as it relies on the expert reviewer's personal judgment and communication style. This can lead to variability in the feedback provided to different trainees or for different scans, potentially affecting the consistency and fairness of the learning experience.

Furthermore, the feedback process is typically conducted in the language of the expert reviewer, which may not be the native language of the trainee. This can pose a barrier to effective communication and understanding, particularly in global healthcare settings where practitioners may come from diverse linguistic backgrounds.

As a result, there is a need for a streamlined, automated, and standardized review process for ultrasound data.

### SUMMARY

This Summary is provided to introduce a selection of concepts in a simplified form that are further described below in the Detailed Description. This Summary is not intended to identify key features or essential features, nor is it intended to limit the scope of the claims included herewith.

Embodiments described in the present disclosure relate to a system and method for generating feedback on ultrasound scans. In particular, the present disclosure may provide a system and method that utilizes an artificial intelligence (AI) model to generate high-yield, clinically useful, and individualized reviews and feedback on any type of ultrasound scans performed by healthcare practitioners. This system and method may address the challenge of providing timely, accurate scan review and quality assessment in the field of ultrasound diagnostics, which is often time-consuming and requires expert knowledge. For example, embodiment disclosed herein relate to a system for generating feedback on ultrasound scans, comprising: an ultrasound scan dataset generated by an ultrasound scan device; an artificial intelligence (AI) model trained on a diverse dataset of ultrasound scans; a set of assessment facts provided by human reviewers and other automated sources used as the basis of a review of the ultrasound scan dataset; a set of instructions for the AI model detailing the desired format and writing style for the review of the ultrasound scan dataset, and providing background medical information for reference; and a user interface driven by a per-exam configuration, allowing a reviewer to select appropriate items based on findings accuracy, scan completeness, and other teaching points. In one embodiment, the per-exam configuration may comprise data specific to a single exam or set of exams, wherein the data comprises any of (a) a set of standardized sonographic findings, (b) scan completeness criteria, (c) discussion points, (d) teaching points, and (e) technical recommendations.

Aspects of the present disclosure relate to a system for generating feedback on ultrasound scans including an artificial intelligence (AI) model trained on a diverse dataset of ultrasound scans and associated findings. This diversity in the training data may enable the AI model to understand and interpret scans produced by different ultrasound machines, thereby enhancing its versatility and applicability across a wide range of clinical settings. For example, the AI model may be capable of generating high-yield, clinically useful, individualized reviews and feedback on any type of ultrasound scans performed by healthcare practitioners. It is contemplated that the system may streamline, automate, and standardize the scan review process, thereby reducing the time and effort traditionally associated with manual scan reviews by experts.

According to an aspect of the present disclosure, the system may comprise a set of assessment facts used as the basis of the review. The assessment facts may be provided by human reviewers, automated sources, or combinations thereof. These facts may relate to any of accuracy, completeness, and educational points, among others. In accordance with one embodiment, the system may comprise a set of instructions for the AI model, detailing the desired format and writing style for the review, and providing background medical information for reference.

According to another aspect of the present disclosure, the system may comprise a user interface driven by the per-exam configuration, allowing the reviewer to select appropriate items based on findings accuracy, scan completeness, and other teaching points. For instance, the user interface may provide options for the reviewer to indicate whether the scan has accurately captured the target anatomy, whether all the requisite views have been included, and whether there are any specific educational points that the feedback should address. The system may then provide this information to the AI model for text generation, which synthesizes the information and generates the desired written review output.

According to other aspects of the present disclosure, the system may comprise additional features, such as the ability to generate reviews in multiple languages. It is contemplated that generating reviews in multiple languages may enhance the accessibility of the system for non-native English speakers. The system may also include power management features to optimize its performance and energy efficiency, and safety features to protect the integrity of the ultrasound scans and the privacy of the patients.

According to yet another aspect of the present disclosure, the system may be adaptable to a variety of healthcare settings, including hospitals, clinics, and field environments. The system may take into account the different workflows and requirements that may be present in each setting. This may provide reliable and timely feedback to healthcare practitioners regardless of their location.

In some embodiments, the AI model is a large language model trained on a diverse dataset of ultrasound scans. The large language model may be capable of synthesizing the assessment facts and instructions to generate the desired written review output. In one embodiment, the large language model may be capable of generating the desired written review output in a plurality of languages. In an embodiment, the set of assessment facts comprises binary and categorical facts related to the accuracy and completeness of the ultrasound scans.

In one embodiment, the set of instructions for the AI model comprises one or more examples of desired output and detailed information on proper ultrasound procedures. In some embodiments, the user interface driven by the per-exam configuration allows the reviewer to select items based on findings accuracy, scan completeness, and other teaching points. In an embodiment, the system further comprises a feature that allows the generation of reviews in multiple languages.

Embodiments disclosed herein also relate to a method for generating feedback on ultrasound scans, comprising the steps of: receiving ultrasound scan data; processing the scan data using an artificial intelligence (AI) model trained on a diverse dataset of ultrasound scans and associated findings; generating a set of assessment facts based on the processed scan data; providing the assessment facts and a set of instructions to the AI model; and generating a written review of the ultrasound scan based on the assessment facts and instructions.

In some embodiments, the method further includes a step of delivering the written review of the ultrasound scan to the healthcare practitioner via a selected delivery method.

Embodiments disclosed herein further relate to a computer-readable medium storing instructions that, when executed by a processor, cause a system to: receive ultrasound scan data; process the scan data using the AI model trained on a diverse dataset of ultrasound scans and associated findings; generate a set of assessment facts based on the processed scan data; provide the assessment facts and a set of instructions to the AI model; and generate a written review of the ultrasound scan based on the assessment facts and instructions.

In some embodiments, the diverse dataset of ultrasound scans and associated findings comprise data from various types of ultrasound machines and different healthcare settings. The diverse dataset may further comprise data from different types of ultrasound scans, including 2D, 3D, and Doppler scans. In one embodiment, the set of assessment facts comprises data related to any of the accuracy of the ultrasound scan, the completeness of the scan, and educational points derived from the scan.

In an embodiment, the set of instructions for the AI model comprises guidelines for generating the written review in a specific language selected by the user. In some embodiments, the user interface driven by the per-exam configuration comprises options for the reviewer to select teaching points based on the specific type of ultrasound scan being reviewed.

It is an object of the system for generating AI-powered feedback on ultrasound scans to offer a solution to the time-intensive and expert-dependent process of manual scan reviews. The system's adaptability, versatility, and user-friendly design are contemplated to make it a valuable tool for healthcare practitioners in a wide range of specialties and settings.

It is contemplated that, in some embodiments, the system may quickly create high-yield, clinically useful, individualized expert reviews and feedback on any type of ultrasound scans, exponentially reducing the time and effort required. For example, the generation of detailed ultrasound scan quality reviews may be based on a limited number of interactions between a user and the system. In some embodiments, the system may generate written reviews in a consistent style, such as encouraging or empathetic. In additional embodiments, the system may enhance fluency of English language reviews by non-native speakers with limited English language skills and may enable the generation of reviews in other languages where the reviewee is not fluent.

Additional aspects related to this disclosure are set forth, in part, in the description which follows, and, in part, will be obvious from the description, or may be learned by practice of this disclosure.

It is contemplated that the system and method of the present disclosure may offer several benefits. For instance, it may streamline, automate, and standardize the scan review process, thereby reducing the time and effort typically associated with manual review by experts. It may also enhance the fluency of English language reviews by non-native speakers and enable the generation of reviews in other languages. Furthermore, it may provide feedback that is tailored to the specific type of ultrasound scan and the level of training of the healthcare practitioner, thereby improving the quality of care and patient safety.

It is to be understood that both the forgoing and the following descriptions are exemplary and explanatory only and are not intended to limit the claimed disclosure or application thereof in any manner whatsoever.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is a block diagram of a system for generating feedback on ultrasound scans, according to an embodiment of the present disclosure.

FIG. 2 is a process flowchart for Feedback Generation System, according to an embodiment of the present disclosure.

FIG. 3 illustrates an embodiment of the AI Model Architecture.

FIG. 4 illustrates an embodiment of the Sequence Diagram for System Component Interaction.

FIG. 5 illustrates an embodiment of the User Interface Driven by Per-Exam Configuration, according to an embodiment of the present disclosure.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

Embodiments described in the present disclosure relate to a system and method for generating feedback on ultrasound scans. In particular, the present disclosure may provide a system and method that utilizes the AI model to generate high-yield, clinically useful, and individualized reviews and feedback on any type of ultrasound scans performed by healthcare practitioners.

More specifically, the system and method of the present disclosure may comprise an AI model trained on a diverse dataset of ultrasound scans and associated findings. The diverse dataset may include data from various types of ultrasound machines, which may encompass a range of manufacturers, models, and technical specifications. In an embodiment, the diverse dataset may also include data from different healthcare settings. These settings may range from large hospitals and specialized clinics to primary care facilities and field settings. By training the AI model on data from these varied settings, the system may be able to generate feedback that is relevant and applicable to the specific context in which the ultrasound scan was performed. In some embodiments, the diverse dataset further includes data from different types of ultrasound scans. These may include 2D scans, which provide two-dimensional grayscale images of the body's internal structures; 3D scans, which offer volumetric data for a more comprehensive view of the anatomy; and Doppler scans, which visualize blood flow within vessels or heart chambers. By training the AI model on these different types of scans, the system may be capable of generating feedback that is tailored to the specific type of scan, thereby enhancing the accuracy and relevance of the feedback.

The training of the AI model on the diverse dataset may enable it to understand and interpret a wide range of ultrasound scans, thereby enhancing its ability to generate accurate and useful feedback. It is contemplated that the AI model may be capable of evaluating numerous data points in the ultrasonic scan, which is permitted by the AI models'ability to compare and evaluate the data captured by the ultrasound scan.

The AI model may be a large language model capable of synthesizing a set of assessment facts and a set of instructions to generate the desired written review output. The set of assessment facts may comprise any of binary and categorical facts related to the accuracy and/or completeness of the ultrasound scans. For instance, the assessment facts may indicate whether a particular anatomical structure has been accurately captured in the scan, or whether all the requisite views have been included in the scan. These assessment facts may serve as the basis for the review, providing the AI model with specific information about the scan that can be used to generate the feedback. The set of instructions for the AI model may comprise a desired format and/or writing style for the review, as well, in some embodiments, as background medical information for reference. The desired format and writing style may specify how the feedback is to be structured and presented, while the background medical information may provide the AI model with a context for interpreting the scan and generating the feedback. For example, the instructions may specify that the feedback is to be written in a supportive and constructive tone and may provide reference texts on ultrasound techniques and findings for the AI model to draw upon.

Synthesizing the assessment facts and instruction may comprise integrating the assessment facts, which provide specific information about the scan, with the instructions, which guide the format and content of the review. The large language model may use advanced AI techniques and algorithms, such as deep learning and natural language processing, to perform this synthesis and generate the written review.

In addition, the system and method may include a user interface driven by per-exam configuration, allowing the reviewer to select appropriate items based on findings accuracy, scan completeness, and other teaching points for a specific exam and/or plurality of exams. The system and method may also provide the capability to generate reviews in multiple languages, enhancing its accessibility to healthcare practitioners around the world. The ability to generate feedback in multiple languages may be particularly beneficial in global healthcare settings, where practitioners may have varying levels of proficiency in different languages.

In some embodiments the method or methods described herein may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above-described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

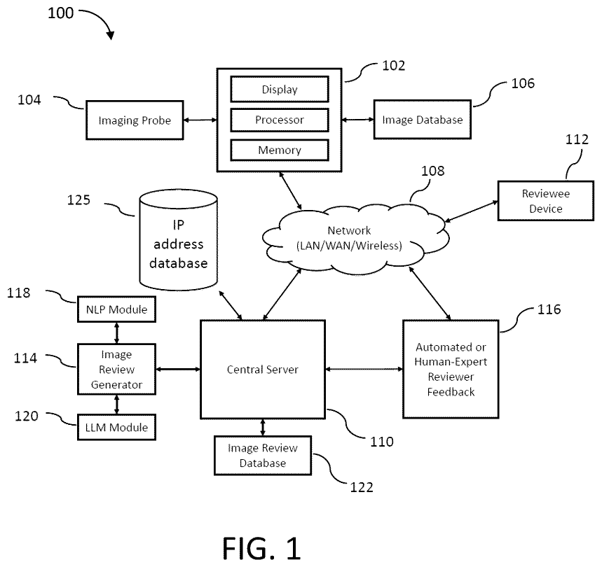

FIG. 1 shows a block diagram of the architecture of a system 100 for generating feedback on ultrasound scans. In some embodiments, system 100 is enabled to access an IP address database 125 to provide secure communication between the system 100 and a central server 110. The system 100 comprises an ultrasound device 102 reversibly connected to an ultrasound imaging probe 104 and an ultrasound scan image database 106. The system 100 communicates through a network 108 communicatively coupled to the central server 110, an automated or human-expert reviewer feedback module/interface 116, an image review generator 114 and a reviewee device 112. The central server 110 coordinates the data flow, interfacing with the image review generator 114 which comprises an NLP module 118, and an LLM module 120, and an image review database 122. Various elements shown in the figure are described in more detail herein.

The ultrasound device 102 may be a medical imaging device designed to generate ultrasound waves and capture the reflected waves to create images of structures within the body and is equipped with a processor, a memory, and a display. The processor in the ultrasound device 102 may be responsible for controlling the operation of the ultrasound device 102, processing the captured ultrasound data, and generating the ultrasound images. The memory stores the ultrasound data and the generated images, while the display presents the images for review. The ultrasound device 102 may, in one embodiment, be integrated with the ultrasound imaging probe 104 into a single handheld unit, simplifying the setup and making the system more portable and convenient for point-of-care use.

However, in another embodiment, the ultrasound device 102 and the ultrasound imaging probe 104 may be independent components in communication with one another. For example, the ultrasound imaging probe 104 may be a handheld device that is connected to the ultrasound device 102. In one embodiment, the ultrasound device 102 could be wirelessly connected to the ultrasound imaging probe 104, eliminating the physical connection and providing more flexibility in the positioning and movement of the probe during the scan. In another embodiment, the ultrasound device 102 could be in wired communication with the ultrasound imaging probe 104. The ultrasound imaging probe 104 may comprise piezoelectric crystals that generate ultrasound waves when an electric current is applied. The ultrasound imaging probe 104 may be used to transmit the ultrasound waves into the body and receive the reflected waves, which are then processed by the ultrasound device 102 to create images.

The ultrasound scan image database 106 may be a digital repository that stores ultrasound scan images. The ultrasound scan image database 106 may comprise images from various types of ultrasound scans and may be used for training the AI model, comparing new scans with previous ones, or archiving scans for future reference. In an embodiment, the ultrasound scan image database 106 could be cloud-based, allowing for remote storage and access of the scan data, and facilitating collaboration and consultation among healthcare practitioners in different locations. The ultrasound scan image database 106 may comprise advanced search and indexing features, enabling quick and efficient retrieval of specific scans or sets of scans based on various criteria such as patient ID, scan date, or diagnostic findings.

The network 108 refers to a digital communication system that allows data to be transferred between different components of the system 100. The network 108 may enable the ultrasound device 102 to communicate with the central server 100 and other components of the system 100. In some embodiments, the network can be a Local Area Network (LAN), Wide Area Network (WAN), or a wireless network. In one embodiment, the network 108 could be a secure private network, ensuring the privacy and security of the transmitted data.

The central server 110 may comprise a computer system that manages and coordinates the data flow within the system 100. The central server 110 may interface with the image review generator 114 and the image review database 122, to ensure comprehensive data management and feedback generation. The central server 110 may be a distributed server system, enhancing the system's scalability and reliability.

In one embodiment, the reviewer feedback module/interface 116 allows a reviewer, either a human expert or an automated AI, to interact with the system 100 and provide feedback on the ultrasound scans. The reviewer feedback module/interface 116 may include user-friendly features such as drag-and-drop functionality, voice recognition, or touch screen compatibility, facilitating the review process and enhancing user experience. In one embodiment, the reviewer feedback module/interface 116 may be at least partially automated. In an embodiment, the reviewed feedback module/interface 116 may be at least partially dependent on a human-expert.

The image review generator 114 may process the ultrasound images and generate feedback based on the analysis using AI algorithms. In an embodiment, the image review generator 114 could be equipped with machine learning capabilities, enabling it to learn from the feedback provided by human experts and continuously improve its performance.

The image review generator comprises an NLP module 118 and an LLM module 120. In an embodiment, the NLP module 118 is a component of the image review generator 114 that uses Natural Language Processing (NLP) techniques to understand and interpret the language-based elements of the ultrasound scans, such as annotations or labels. In some embodiments, the NLP module 118 could be equipped with multilingual capabilities, enabling it to understand and generate feedback in multiple languages. It is contemplated that this may enhance the accessibility of the system to healthcare practitioners around the world, allowing them to receive feedback in their preferred language.

The LLM Module 120 may be a component of the image review generator 114 that comprises a large language model (LLM) trained on a diverse dataset of ultrasound scans and associated findings. The LLM module 120 is capable of synthesizing a set of assessment facts and a set of instructions to generate the desired written review output. It is contemplated that in some embodiments, the LLM module 120 could be trained on additional datasets, such as medical textbooks or expert commentaries, enhancing its knowledge base and improving the quality of the generated feedback.

In some embodiments, the information provided to large language model for text generation includes (a) general instructions, (b) background information (e.g.: reference material on proper gallbladder scanning technique), (c) sample reviews demonstrating desired writing style, (d) a summary of findings (review facts) based on reviewer information entered into user interface, and (e) optionally desired output language.

The use of the LLM for review generation may yield any of the following benefits: (a) LLM is a powerful technique for controlling writing style, including factors such as encouragement, empathy, (b) merger of review facts with background material enables LLM to synthesize text unique to the individual review, (c) natural run-to-run text generation variability produces bespoke rather than canned reviews, even for the same set of review input facts.

In one embodiment, the LLM utilizes a variety of advanced AI techniques and algorithms. These include, but are not limited to, deep learning neural networks, natural language processing (NLP), and machine learning algorithms such as transformer models, which are known for their ability to handle sequential data and their attention mechanisms that weigh the relevance of different parts of the input data. The LLM model 120 may be trained on a vast corpus of medical texts and ultrasound imaging data, enabling it to understand and generate contextually relevant and medically accurate feedback.

In some embodiments, the LLM model 120 employs reinforcement learning techniques to fine-tune its outputs based on user interactions and feedback, ensuring continuous improvement in the quality and accuracy of the generated reviews. In one embodiment, the image review generator 114 uses prompt engineering with existing LLMs. In some embodiments, image review generator 114 uses examples of properly structured reviews provided by humans or edited/modified by humans to aid the text generation using an LLM. In a further embodiment, RAG (retrieval augmented generation) is used for text generation.

In one embodiment, the image review database 122 is a digital repository that stores the generated reviews for future reference. The image review database 122 may ensure comprehensive data management and facilitate the generation of feedback based on historical data. In an embodiment, the image review database 122 may be integrated with an electronic health record (EHR) system, enabling the feedback to be automatically incorporated into the patient's medical record.

The reviewee device 112 may be a digital device, such as a computer, tablet, or smartphone, used by the healthcare practitioner to receive and review the feedback. The reviewee device 112 allows the practitioner to access the feedback in a convenient and user-friendly manner.

It is contemplated that the system 100 is designed to comply with relevant healthcare regulations, such as HIPAA in the United States, which sets standards for the protection of sensitive patient data. The privacy and security of patient data may be ensured through several robust mechanisms. Firstly, the security and privacy are strengthened by introduction of a verification protocol wherein the central server registers and maintains a list of approved ultrasound imaging devices based on the device ID or the device IP address to be included in the IP address database 125.

In some embodiments, the system may be configured to ensure that the data provided is an authorized scan. In an embodiment, the system verifies that the ultrasound scan received by the system 100 is from a sanctioned source, such as an ultrasound device 102, that has been previously authorized to communicate with the system. The system may, in some embodiments, capture the Internet Protocol Address (IP address) from the source providing the scan and compares the IP address to a database of IP addresses that have been previously identified by the system administration operators.

For example, the central server 110 may compare the sending device ID or IP address to the IP address database 125 of approved device ID or device IP addresses. If the sending device ID or IP address is also recorded in the database of IP addresses the system 100 allows the ultrasound scan to be uploaded to the system 100 to be evaluated by the AI model. If the sending device IP address is not in the database, the system does not allow the ultrasonic scan to be uploaded to the system. It is contemplated that this may enhance the system's security and protect the system 100 from fraudulent scans and unauthorized access.

In some embodiments, the privacy and security of patient can further be ensured by anonymizing all patient data before being processed by the AI model, ensuring that no personally identifiable information is used during the feedback generation process. In addition, the system 100 may employ state-of-the-art encryption protocols to protect data during transmission and storage, preventing unauthorized access. Further, access to the feedback and underlying patient data may be strictly controlled through a role-based access control system, ensuring that only authorized personnel can view sensitive information. In some embodiments, regular security audits and updates are conducted to maintain the integrity of the system and to adapt to emerging security threats.

In an embodiment, the system 100 may be integrated with other systems or technologies to enhance its functionality. For example, the system may be integrated with the EHR system as disclosed herein. This integration may allow the system 100 to automatically incorporate patient information into the feedback, providing a more comprehensive and personalized review of the ultrasound scan. By accessing relevant patient data, such as medical history or previous imaging studies, the system 100 may be able to provide feedback that is not just technically accurate but also clinically relevant, enhancing the value of the feedback for the healthcare practitioner.

The system 100 may also be integrated with other diagnostic tools. It is contemplated that this integration may enable the system 100 to provide a more comprehensive review of the patient's condition. For example, the system may be integrated with a computed tomography (CT) scanner or a magnetic resonance imaging (MRI) machine, allowing the system to compare the ultrasound scan with other imaging studies. This may provide a more holistic view of the patient's condition, enabling the system to generate feedback that takes into account a wider range of diagnostic information. The integration with other diagnostic tools may enhance the system's utility as a comprehensive diagnostic support tool, aiding healthcare practitioners in making more informed medical decisions.

In some embodiments, the system 100, illustrated in FIG. 1, generates a set of assessment facts based on the processed scan data. These assessment facts may include binary and categorical facts related to the accuracy and completeness of the ultrasound scans. For instance, binary facts may indicate whether a particular anatomical structure has been accurately captured in the scan or not, while categorical facts may classify the level of completeness of the scan, such as whether all the requisite views have been included or not. These assessment facts may serve as the basis for the review, providing an AI model with specific information about the scan that can be used to generate the feedback.

The set of assessment facts may also include data related to educational points derived from the scan. These educational points may provide additional context for the feedback, highlighting areas where the practitioner can improve their scanning technique or interpretation skills. For example, an educational point may note that a particular view of the heart was not adequately visualized in the scan, suggesting that the practitioner may benefit from additional training on cardiac ultrasound techniques. In some embodiments, the set of assessment facts are generated automatically by the AI model based on its analysis of the scan data. Alternatively, the assessment facts may be provided by human reviewers, who may use their expert knowledge to evaluate the scan and identify areas of interest or concern. The system may use a combination of AI-generated and human-provided assessment facts, leveraging the strengths of both approaches to provide comprehensive and accurate feedback on the ultrasound scans.

In some embodiments, the system 100 may be designed with different power management features to optimize its performance and energy efficiency. For instance, the system 100 may be designed to enter a low-power mode when not in use, thereby conserving energy. This could be particularly beneficial in healthcare settings where the system 100 is used intermittently, allowing for energy savings during periods of inactivity.

The system 100 may prioritize power usage based on the complexity of the ultrasound scans being reviewed. For example, the system may allocate more processing power for analyzing complex scans, such as 3D or Doppler scans, which may require more computational resources to process and interpret. Conversely, the system may allocate less power for simpler scans, such as 2D scans, thereby optimizing energy usage based on the demands of the task at hand.

In some embodiments, the system 100 includes power-saving features, such as automatic shutdown or sleep mode, after a period of inactivity. It is contemplated that this could prevent unnecessary power consumption when the system 100 is not in use, further enhancing its energy efficiency.

The system 100 may also be designed to operate efficiently under different power supply conditions. For instance, the system 100 may be designed to operate on battery power in field settings or during power outages, ensuring uninterrupted operation even in challenging conditions.

The system 100 may also comprise features for monitoring and managing its power usage. For example, the system 100 may provide alerts or notifications when the power level is low, or when the power usage exceeds a specified threshold. This could help ensure that the system 100 operates within its power capacity, preventing overloads or disruptions due to power issues. These power management features may enhance the system's operational efficiency and sustainability, making it a practical and eco-friendly solution for generating feedback on ultrasound scans in a wide range of healthcare settings.

In some embodiments, the system 100 may be equipped with various safety features to protect the integrity of the ultrasound scans and the privacy of the patients. For instance, the system 100 may include encryption features to secure the ultrasound scans during transmission and storage. This could prevent unauthorized access to the scan data, ensuring that the scans are protected from tampering or misuse. The system 100 may also include access control features to restrict who can view the scans and the feedback. For example, the system 100 may require user authentication, such as a password or biometric verification, before granting access to the scan data or feedback. This could prevent unauthorized access to sensitive patient information, ensuring that the privacy of the patients is protected.

In some embodiments, the system 100 includes features for monitoring and logging access to the scan data and feedback. For instance, the system 100 may keep a record of who accessed the scan data or feedback, when they accessed it, and what actions they performed. This could provide an audit trail for tracking and managing access to the scan data and feedback, further enhancing the security of the system.

The system 100 may also be designed to comply with relevant healthcare regulations, such as the Health Insurance Portability and Accountability Act (HIPAA) in the United States, which sets standards for the protection of sensitive patient data. This could ensure that the system adheres to legal and ethical standards for data privacy and security, providing assurance to healthcare practitioners and patients alike.

In some embodiments, the system 100 is designed with various customization and personalization features to cater to the preferences and requirements of different users. For instance, the system 100 may allow users to customize the types of feedback they receive. This could include any of selecting the level of detail in the feedback, the format of the feedback, or the specific areas of focus in the feedback. For example, a novice user may prefer feedback that is more basic and educational, focusing on foundational aspects of ultrasound scanning and interpretation. An expert user, on the other hand, may prefer more advanced, nuanced feedback that delves into complex findings and subtleties of the scan. By allowing users to customize the feedback, the system 100 may ensure that each healthcare practitioner receives the information that is the most appropriate and beneficial for their professional development.

It is contemplated that these safety features may enhance the trustworthiness and reliability of the system 100, thus creating a secure and dependable tool for generating feedback on ultrasound scans in various healthcare settings.

The system 100 may also include features for saving and managing user preferences. For example, the system 100 may include a user profile feature that allows users to save their preferences and apply them to future scan reviews. It is contemplated that this could save users time and effort in setting the user's preferences for each scan review and ensure that the feedback is consistently tailored to the user's preferences. The system 100 may also include a preference management feature that allows users to update or change their preferences as their skills and requirements evolve. This could ensure that the system 100 remains adaptable and responsive to the changing learning and clinical demands of the healthcare practitioners.

It is contemplated that the customization and personalization features may enhance the flexibility and/or user-friendliness of the system 100, making it a versatile and adaptable tool for generating feedback on ultrasound scans in a wide range of healthcare settings.

The system 100 may be designed to identify and address inaccuracies or errors in ultrasound scans through a multi-faceted approach. Initially, the AI model may cross-reference the uploaded scan data with a comprehensive database of sonographic images and findings to detect any discrepancies or anomalies. If an inaccuracy or error is detected, the system 100 flags the specific area of concern and provides detailed feedback to the healthcare practitioner. This feedback may include suggestions for rescanning, if possible, or guidance on interpreting the scan with the noted limitations.

Additionally, the system 100 may incorporate a feedback loop where human experts, such as a certified radiologist, can review and correct the AI-generated feedback, which in turn is used to further train and refine the AI model's accuracy. The system 100 may also provide statistical confidence levels for its findings, allowing practitioners to gauge the reliability of the feedback and make informed decisions accordingly. In some embodiments, the LLM may be used to self-review the quality and accuracy of the generated output, and provide corrections as needed. Example of self-review may include: (a) ensuring that statements in review are in alignment with directly provided to the model, either as facts (assertions) from the reviewer or background context; and (b) ensuring that the style of generated review is consistent with desired output standards.

The system 100 may be optimized to handle a wide array of medical conditions or anomalies that may appear in ultrasound scans by leveraging its extensive training on a diverse set of medical imaging data and diagnostic outcomes. An AI model may be designed to recognize and categorize various sonographic patterns and features associated with different pathologies. When a scan is uploaded, the system 100 analyzes the images using advanced image recognition algorithms to identify potential conditions or anomalies. It may then cross-reference these findings with its database to provide a differential diagnosis or highlight areas of concern. In one embodiment, the feedback includes detailed descriptions of the identified conditions or anomalies, along with recommendations for further evaluation or follow-up as appropriate. This approach allows the system to assist healthcare practitioners in making more informed decisions and improving diagnostic accuracy, regardless of the type or complexity of the medical condition presented in the scan.

In an embodiment, the system 100 may be designed to identify and provide feedback on various ultrasound scan artifacts, such as shadowing, enhancement, and reverberation, which can affect the interpretation of the scan. The AI model may be trained on a dataset that includes examples of these common artifacts, enabling it to recognize their presence in the scan data. When an artifact is detected, the system 100 provides feedback that describes the artifact, explains its potential impact on image interpretation, and offers guidance on how to differentiate artifacts from true pathological findings.

The system 100 may, in an embodiment, be optimized to address various complications that may arise during ultrasound scanning, such as patient movement and poor image quality. When such complications are reported by reviewers, the feedback includes actionable recommendations for adjusting the scan parameters or environment to improve image quality. Additionally, the system 100 can, in some embodiments, guide the healthcare practitioner on how to interpret images with known quality issues, ensuring that the diagnostic process can continue even when ideal scanning conditions are not met. This proactive approach to handling complications enhances the utility of the ultrasound scans and supports practitioners in achieving the best possible outcomes.

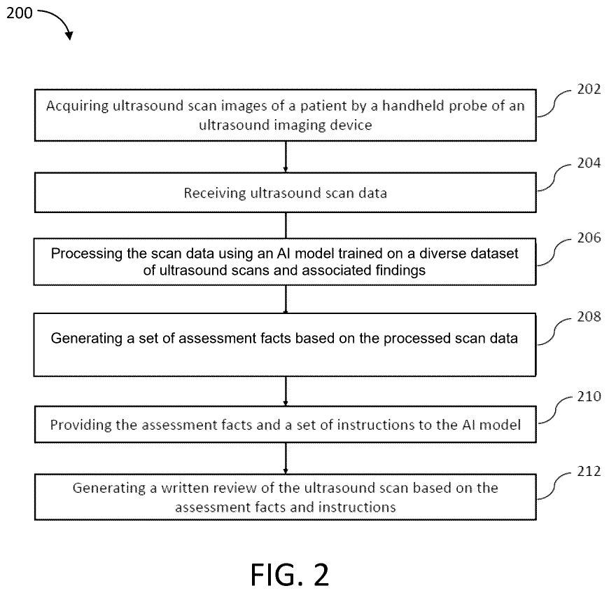

FIG. 2 depicts a flowchart illustrating the overall process 200 of the system for generating feedback on ultrasound scans. As illustrated, the process 200 comprises steps such as receiving ultrasound scan data 204, processing the data with the AI model 206, generating assessment facts 208, providing the assessment facts and a set of instructions to the AI model 210, and generating the written review 212.

In step 202 ultrasound scan data of a patient are acquired using a handheld probe of an ultrasound imaging device. The ultrasound scan may comprise any of images, videos, and any associated metadata from the scanning device, is contemplated. Of course, other manners of acquiring ultrasound scan images of a patient are contemplated. In step 204, the ultrasound scan data is received by the system. The ultrasound scan data may include images or videos captured during the ultrasound examination, as well as any annotations or measurements made by the healthcare practitioner. The scan data may be received from a variety of sources, such as ultrasound machines, handheld ultrasound devices, and/or digital storage devices.

In step 206, the AI model processes the received ultrasound scan data. This may comprise analysis of the input data using an AI model utilizing algorithms to identify relevant features, patterns, and anomalies within the ultrasound scans. In one embodiment. the AI model, which may be a large language model trained on a diverse dataset of ultrasound scans and associated findings, may analyze the scan data to identify relevant features and patterns. This analysis may involve advanced image recognition algorithms that can interpret the visual content of the scan, as well as machine learning algorithms that can recognize patterns and correlations in the scan data.

Following step 206, the system may generate assessment facts in step 208 from the processed data. It is contemplated that the assessment facts may be provided by human reviewers, automated sources, or a combination thereof. At this step, the system may generate objective facts about the ultrasound scan data, such as the presence or absence of specific features, the quality of the images, and any deviations from standard scanning protocols. In some embodiments, the assessment facts may be categorical assessment facts, binary assessment facts, or combinations thereof. For instance, binary facts may indicate whether a particular anatomical structure has been accurately captured in the scan, while categorical facts may classify the level of completeness of the scan, such as whether all the requisite views have been included or not. In one embodiment, the assessment facts may comprise any of accuracy, completeness, education points, or other facts that may be contemplated. The assessment facts may serve as the basis for the review, providing the AI model with specific information about the scan that can be used to generate the feedback.

In step 210, the system may provide the AI model with a set of instructions for review generation and the assessment facts. In one embodiment, the set of instructions may be a set of instructions comprising a desired format and writing style for the review, such as one or more examples of the desired review. In an embodiment, the set of instructions may comprise a medical context for the review, comprising background medical information, such as reference text, which provide detailed information on any of proper procedures, findings, interpretations, pitfalls, and clinical integration.

In some embodiments, the AI model may be an AI LLM model that, when provided with the assessment facts and the set of instructions for review generation, is capable of synthesizing the received information and generating the desired written review output.

In some embodiment, the set of instructions for the AI model includes one or more examples of desired output and detailed information on proper ultrasound procedures. For example, the set of instruction may comprise reference texts on ultrasound techniques and findings, as well as guidelines on how to perform specific types of ultrasound scans. By providing this background information, the AI model may generate feedback that is not just technically accurate but also clinically relevant, enhancing the educational value of the feedback for the healthcare practitioner.

Further, the set of instructions may guide the AI model in generating the written review output, providing it with a framework for structuring the feedback and a reference for interpreting the ultrasound scans. For instance, the instructions may specify that the feedback is to be written in a supportive and constructive tone and may provide reference texts on ultrasound techniques and findings for the AI model to draw upon. This may ensure that the feedback is not just technically accurate but also pedagogically effective, fostering a positive learning environment for the healthcare practitioners.

In one embodiment, the set of instructions for the AI model may be customizable, allowing the user to specify the desired format, writing style, and language for the feedback. This is contemplated to enhance the flexibility of the system, enabling it to cater to the specific preferences and requirements of different healthcare practitioners. For instance, a novice practitioner may prefer feedback in a simple, easy-to-understand language, while an experienced practitioner may prefer more detailed and technical feedback. Similarly, a practitioner who is a non-native English speaker may prefer feedback in their native language. By allowing the user to customize the instructions for the AI model, the system may ensure that the feedback is both useful and accessible to a wide range of healthcare practitioners.

In an embodiment, the set of instructions for the AI model may comprise guidelines for generating the written review in a specific language selected by the user. It is contemplated that this may enhance the accessibility of the system to healthcare practitioners around the world, allowing them to receive feedback in their preferred language. The AI model may be equipped with advanced language processing capabilities, enabling it to understand and generate text in different languages. This may be beneficial in global healthcare settings, where practitioners may have varying levels of proficiency in different languages. By providing feedback in the practitioner's preferred language, the system may facilitate better understanding and application of the feedback, thereby enhancing the practitioner's ultrasound interpretation skills and patient care outcomes.

At step 212, a written review of the ultrasound scan based on the assessment facts and instructions is generated. The written review may be a text document or a written report that provides a detailed analysis of the ultrasound scan, including an evaluation of the scan's accuracy and completeness, as well as educational points derived from the scan. In one embodiment, the AI model generates the written review based on the assessment facts and instructions. For example, the AI model synthesizes the assessment facts with the provided instructions to create a coherent, written review of the ultrasound scan. The written review may be delivered to the healthcare practitioner (the reviewee) through various methods such as email, electronic health records, or a direct interface within the system.

In some embodiments, following generating the written review at step 212, the review may be delivered to a user, such as a health practitioner, via a selected delivery method. This delivery method may be selected based on the practitioner's preferences and the specific requirements of the clinical or educational setting. For instance, the feedback may be delivered via email, allowing for easy archiving and reference. Alternatively, the feedback may be delivered via SMS notifications, providing quick, on-the-go updates that can alert practitioners to review their feedback at their earliest convenience. The feedback may be delivered via app notifications, serving as a seamless way to integrate feedback within the healthcare practitioner's workflow, especially when using mobile devices or tablets. Each delivery method ensures that feedback is not just timely but also accessible in a manner that aligns with the practitioner's work habits and technology usage, facilitating prompt review and application of the feedback.

While reference is made to a written review, any form of feedback may be generated. For example, the feedback may be generated as an audio recording. It is contemplated that this could be particularly useful for practitioners who prefer auditory learning or are visually impaired. The system may utilize text-to-speech technology to convert the written feedback into an audio format. The audio feedback may, in some embodiments, be generated in a specific language selected by the user, further enhancing the system's versatility and global applicability.

In some embodiments, the feedback may be delivered as a visual presentation. This could include annotated images or clips from the ultrasound scan, with voiceover explaining the findings. The visual presentation may be particularly beneficial for visual learners and for demonstrating scanning techniques or specific findings. The visual feedback may be generated in a specific language selected by the user, ensuring that the feedback is accessible and effective for enhancing their ultrasound interpretation skills.

In some embodiments, the feedback may be delivered in real-time during the ultrasound scan. This could provide immediate guidance to the healthcare practitioner, allowing them to adjust their scanning technique or interpretation on the spot. Real-time feedback may be particularly beneficial in educational settings or in acute care situations where immediate feedback is imperative. However, in other embodiments, the feedback may be delivered as a report after the scan. This could provide a comprehensive review of the ultrasound scan, allowing the healthcare practitioner to reflect on their performance and learn from the feedback at their own pace. Post-scan feedback may be particularly useful for practitioners who prefer to review the feedback in detail and incorporate the learning points into their future practice.

In some embodiments, the system 100 may allow the user to select their preferred feedback format. This feature may enhance the flexibility of the system, enabling it to cater to the specific preferences and requirements of different healthcare practitioners. By providing feedback in the practitioner's preferred format, the system 100 may facilitate better understanding and application of the feedback, thereby enhancing the practitioner's ultrasound interpretation skills and patient care outcomes.

In some embodiments, the system 100 of FIG. 1 includes a computer-readable medium storing instructions that, when executed by a processor, cause the system to perform a series of steps, such as the steps described with reference to FIG. 2.

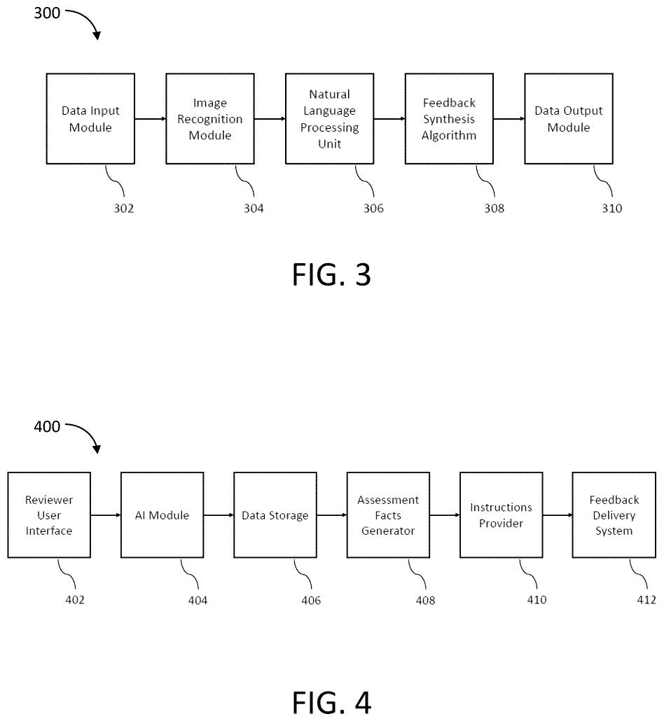

FIG. 3 shows a block diagram of one embodiment of an AI model's architecture 300, showing how it processes input data, such as ultrasound data, and generates assessment facts and written reviews. The block diagram illustrates the internal architecture of the AI model, showing the components involved in processing the ultrasound scan data and generating feedback. As illustrated, the AI model's architecture comprises sub-components such as image recognition modules 304, natural language processing units 306, and feedback synthesis algorithms 308. The AI model's architecture 300 may further comprise a data input module 302 which is the entry point for ultrasound scan data into the AI model. The data input module 302 may receive and organize the ultrasound scan data as it enters the AI system, to ensure it is in the correct format for processing.

The image recognition module 304 may be responsible for analyzing the visual content of the scans. For example, the image recognition module 304 may be a portion of the AI model that applies image analysis techniques to interpret the visual content of the ultrasound scans, identifying anatomical structures and potential abnormalities. The natural language processing (NLP) unit 306 may be a part of the AI model that processes and understands language-based data. It is contemplated that the NLP unit 306 employs language understanding algorithms to process textual data, such as annotations or notes associated with the scans, and to generate the textual content of the review.

The feedback synthesis algorithm 308 may integrate assessment facts with instructions to produce a written review. In an embodiment, the feedback synthesis algorithm 308 combines the insights from the image recognition module 306 and the NLP unit 306 with the assessment facts and instructions to produce a structured written review.

The data output module 310 may output the generated written review. In some embodiment, the data output module 310 may format the generated review as per the specified requirements and prepare the formatted written review for delivery to the user.

In one embodiment, the system may utilize advanced language processing algorithms within the AI model to generate the written review output in multiple languages. These algorithms may be capable of accurately translating the feedback into the desired language, ensuring that the feedback retains its original meaning and context. This feature may be particularly useful for non-native English speakers, who may find it easier to understand and apply the feedback when it is provided in their native language.

FIG. 4 is a sequence diagram 400 which illustrates the interaction between the system components, such as a reviewer user interface 402, an AI model 404, a data storage 406, an assessment facts generator 408, an instructions provider 410, and a feedback delivery system 412, during one embodiment of the generation of feedback including the order of operations and the flow of data. In an embodiment, the reviewer user interface 402 may be where the reviewer interacts with the system. For example, the reviewer user interface 402 may be the front-end component where the reviewer interacts with the system, such as a graphical, command line, and/or menu driven user interface, making selections and inputs that will inform the feedback generation process. The AI module 404 may process data and generate feedback. In one embodiment, the AI module 404 applies artificial intelligence algorithms to analyze the scan data and generate feedback.

The data storage unit 406 may be a repository where scan data, assessment facts, and generated reviews are securely stored and managed within the system. The assessment facts generator 408 may be a process or module within the system that creates a structured set of facts about the ultrasound scan based on the AI model's 404 analysis.

The instructions provider 410 may be a process or module that supplies the AI model 404 with instructions for generating feedback, such as a review. In an embodiment, the instructions provider 410 supplies the AI model 404 with the instructions and/or guidelines that dictate how the feedback is to be structured and presented, such as a preferred delivery method. The feedback delivery system 412 may be a mechanism by which the feedback is delivered to the reviewee, for example, a health practitioner. In some embodiment, the feedback delivery system 412 incorporates the mechanism or service that ensures the completed review is delivered to the healthcare practitioner in according the supplied instructions and through the preferred delivery method.

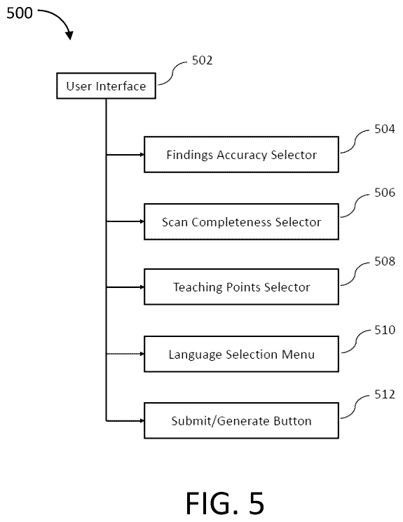

FIG. 5 illustrates a schematic of one embodiment of a user interface 502 driven by per-exam configuration 500, showing how a reviewer can select appropriate items based on findings accuracy, scan completeness, discussion points, teaching points, and technical recommendations, among others. In one embodiment, the per-exam configuration 500 may be specific to a single exam. However, in other embodiments, the per-exam configuration 500 may correspond to a plurality of exams. In some embodiments, the plurality of exams may comprise a shared characteristic, for example any of a scan type, area being scanned, individual being scanned, relevant discussion points, scan completeness, or any other grouping.

In some embodiments, using the user interface 502, the reviewer selects appropriate items basic on findings accuracy, scan completeness, drill-down on issues (e.g.: proper technique for imaging gallstones), sonographic technique recommendations and other teaching points. For example, the user interface driven by per-exam configuration 500 may provide options for the reviewer to indicate whether the scan has accurately captured the target anatomy, whether all the requisite views have been included, and whether there are any specific educational points that the feedback should address. This feature may facilitate a more tailored and relevant feedback process, as the feedback can be customized based on the specific characteristics of each ultrasound scan and the educational goals of the healthcare practitioner.

For example, the user interface of the reviewer feedback module/interface 116 discussed with reference to FIG. 1. Various embodiments and variations regarding a user interface 502 have been described in detail in previous sections. As illustrated in FIG. 5, the user interface 502 may comprise a findings accuracy selector 504, a scan completeness selector 506, a teaching points selector 508, a language selection menu 510, and a submit/generate button 512. In some embodiments, the user interface 502 may be a graphical user interface wherein the user may interact with any aspect of the system through the graphical interface. For example, by selecting a button, drop down menu, text selection, file upload, text input, or other manner of interacting with the graphical user interface.

In one embodiment, the findings accuracy selector 504 is an interface element where the reviewer selects options related to the accuracy of findings. For example, the findings generated in the image review generator 114 discussed with reference to FIG. 1. In an embodiment, the findings accuracy selector 504 is an interactive element within the user interface 502 that allows the reviewer to input and/or select options regarding the accuracy of the findings depicted in the ultrasound scan.

In one embodiment, the scan completeness selector 506 is a graphical display where the reviewer can select options regarding the completeness of the scan. In an embodiment, the scan completeness selector 506 may comprise displaying any of the scan data, such as images or metadata. In some embodiment, the scan completeness selector 506 may receive an input associated with a completeness of the scan, such as whether all the requisite views and structures have been captured.

The teaching points selector 508 may comprise an interface element for selecting teaching points based on the scan. For example, the teaching points selector 508 may be used to select or highlight specific educational points or areas for improvement.

In an embodiment, the user interface 502 driven by the per-exam configuration 500 comprises options for the reviewer to select teaching points based on the specific type of ultrasound scan being reviewed. For example, if the scan is a cardiac ultrasound, the user interface 502 may provide teaching points related to the imaging of the heart's chambers, valves, and blood flow. If the scan is a musculoskeletal ultrasound, the teaching points may pertain to the imaging of bones, muscles, and joints. This feature may ensure that the feedback is not just accurate but also contextually relevant, providing the practitioner with insights that are directly applicable to their specific area of practice.

In some embodiments, the language selection menu 510 may be an interface where the reviewer can choose the language for the feedback. The language selection menu 510 may comprise a dropdown menu or similar interface feature that displays a plurality of language options and allows the reviewer to select one of the plurality of languages. In one embodiment, the user interface 502 may be designed to support multiple languages. This may allow the reviewer to select the language in which the feedback is to be generated, thereby enhancing the accessibility of the system to healthcare practitioners around the world. This feature may enhance the system's versatility and global applicability. It is contemplated that providing feedback in the practitioner's preferred language, the system may facilitate better understanding and application of the feedback, thereby enhancing the practitioner's ultrasound interpretation skills and patient care outcomes.

In an embodiment, responsive to receiving a selection of the plurality of languages, the system may translate any of the report, user interface, and feedback to the desired language. For example, the system may be capable of generating feedback in a wide range of languages, including but not limited to English, Spanish, French, German, Chinese, Japanese, and Arabic.

In an embodiment, the submit/generate button 512 initiates the feedback generation process, discussed with reference to FIG. 2. For example, upon receiving a selection of the submit/generate button 512, such as a click, the system may initiate the feedback generation process, to process the selections made by the reviewer and generate the written review accordingly. While described as a button, any interface may be utilized.

In one embodiment, the reviewee may import a set of still images and/or videos to the review. In an embodiment, the reviewee may import a set of proposed sonographic findings to the review. Of course, the reviewee may import any information to the review, including any of the per-exam type configuration data.

While the embodiments described in the present disclosure are focused on expert written review of ultrasound examinations, the same approach may be used for a broad range of expert reviews, not limited to medicine or human healthcare. For instance, the system may be applied in the field of veterinary medicine, sport medicine, marine animals, educational settings, and research settings. Of course, other uses are contemplated and the aforementioned are provided as non-limiting examples only.

In accordance with one embodiment, a system for generating feedback for at least one ultrasound scan is disclosed, the system comprising:
a. the at least one ultrasound scan represented by ultrasound scan data generated by an ultrasound scan device;
b. an AI model trained on a diverse dataset of stored ultrasound scans;
c. a set of assessment facts provided by human reviewers and other automated sources related to the stored ultrasound scans and used as the basis of a review of the ultrasound scan data;
d. a set of instructions for the AI model detailing the desired format and writing style for the review of the ultrasound scan data created by the AI model, and providing background medical information for reference; and
e. a user interface allowing a reviewer to select appropriate items based on findings accuracy, scan completeness, and other teaching points.

In some embodiments, the AI model is a large language model trained on a diverse dataset of the stored ultrasound scans.

In an embodiment, the large language model synthesizes the set of assessment facts and instructions to generate the desired written review output.

In a further embodiment, the set of assessment facts includes binary and categorical facts related to accuracy and completeness of at least one ultrasound scan.

In one embodiment, the set of instructions for the AI model includes at least one example of desired output and detailed information on proper ultrasound procedures.

In an embodiment, the user interface allows the reviewer to select appropriate items based on findings accuracy, scan completeness, and other teaching points in multiple languages.

In an embodiment, the system further comprises a translator module that generates reviews in multiple languages.

In another embodiment, a method for generating feedback on at least one ultrasound scan is disclosed, comprising the steps of:
f. receiving the at least one ultrasound scan represented by ultrasound scan data from an ultrasound imaging device;
g. processing the ultrasound scan data using an artificial intelligence (AI) model trained on a diverse dataset of stored ultrasound scan data;
h. generating a set of assessment facts based on processed ultrasound scan data;
i. providing the set of assessment facts and a set of instructions to an AI model; and
j. the AI model generates a written review of the at least one ultrasound scan represented by the ultrasound scan data and the written review is based on the set of assessment facts and instructions

In one embodiment of the method, the AI model is a large language model trained on a diverse dataset of stored ultrasound scans.

In an embodiment of the method, the large language model synthesizes the set of assessment facts and instructions to generate a written review in multiple languages.

In an embodiment of the method, the set of assessment facts includes binary and categorical facts related to accuracy and completeness of the ultrasound scan data.

In some embodiments of the method, the set of instructions for the AI model includes one or more examples of desired output and detailed information on proper ultrasound procedures.

In an embodiment of the method, a reviewer uses a user interface to select items based on findings accuracy, scan completeness, and other teaching points.

In some embodiments of the method, further comprising a step of delivering the written review of the at least one ultrasound scan to the healthcare practitioner.

In one embodiment, a computer-readable medium storing instructions that, when executed by a processor, cause a system to:
k. receive at least one ultrasound scan data;
l. process the at least one ultrasound scan data using an artificial intelligence AI model trained on a diverse dataset of stored ultrasound scans and associated findings;
m. generate a set of assessment facts based on processed at least one ultrasound scan data;
n. provide the set of assessment facts and a set of instructions to an AI model; and
o. generate a written review of the at least one ultrasound scan data based on the set of assessment facts and the instructions.

In an embodiment of the computer-readable medium, the diverse dataset of stored ultrasound scans and associated findings includes data from various types of ultrasound machines and different healthcare settings.

In an embodiment of the computer-readable medium, the diverse dataset of stored ultrasound scans further includes data from stored ultrasound scans selected from the group comprising of 2D, 3D, and Doppler scans.

In one embodiment of the computer-readable medium, the set of assessment facts includes data related to the accuracy of the at least one ultrasound scan data, the completeness of the at least one ultrasound scan data, and educational points derived from the at least one ultrasound scan data.

In some embodiments of the computer-readable medium, the set of instructions for the AI model includes guidelines for generating the written review in a specific language selected by the user.

In one embodiment of the computer-readable medium, the user interface includes options for a reviewer to select teaching points based on the specific type of ultrasound scan being reviewed.

The present disclosure provides for a system for generating feedback for at least one ultrasound scan. Such a system may include the at least one ultrasound scan represented by ultrasound scan data generated by an ultrasound scan device. In an embodiment, the at least one ultrasound scan may be stored in the ultrasound scan device, the image database, the network, and/or the central server. Thus, the at least one ultrasound scan may be retrieved directly from the ultrasound scan device or from another component of the system (e.g., the central server, another component connected via the network, and the like). The system may include an image review generator, wherein an AI model trained on a diverse dataset of stored ultrasound scans may be stored in such an image review generator. In further embodiments, the AI model may be stored in any suitable component and its actions may be executed on any sufficient component of the system. For example, in one embodiment, the central server may be configured to execute performance of the AI model. The AI model may contemplate a set of assessment facts provided by human reviewers and/or other automated sources related to the stored ultrasound scans and used as the basis of a review of the ultrasound scan data. The system may include a set of instructions, for example curated by a human administrator, for the AI model detailing the desired format and writing style for the review of the ultrasound scan data created by the AI model, and providing background medical information for reference. However, the instructions may be modified and/or generated by the AI model after initial training. The system may include a user interface allowing a reviewer to select appropriate items based on findings accuracy, scan completeness, and other teaching points. For example, the user interface may be displayed on an end-user facing device, for example, where the end-user is a reviewer or a trainee.

In an embodiment, the AI model is a large language model trained on a diverse dataset of the stored ultrasound scans, wherein the AI model is adapted to assess the efficacy, quality, or other characteristics of said ultrasound scans. In addition to assessing the efficacy, quality, or other characteristics of the ultrasound scans, the large language model may synthesize the set of assessment facts and instructions to generate the desired written review output. In effect, the AI model may both assess the efficacy, quality, or other characteristics of the ultrasound scans while also generating written review output content. In this sense, the AI model may support a dual-role of “grading” the ultrasound scans and “writing up” the ultrasound scans, the write up are one or more teaching points that may be analyzed by the reviewer, reviewee, trainee, etc.

In an embodiment, the system may be configured to track performance of the scan technician trainee. In such an embodiment, the system may store relevant information (e.g., scan feedback, temporal data, number of errors, number of teaching points, number of same errors in light of previous same-error-related feedback, etc.) for a given trainee and/or reviewer. In such a manner, the system may be utilized for tracking the performance of the trainee and/or the efficacy of the reviewer. Accordingly, the scan review generator described herein may be adapted to assess performance of “teacher and student.”

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A system for generating feedback for at least one ultrasound scan, the system comprising:
the at least one ultrasound scan represented by ultrasound scan data generated by an ultrasound scan device;
an AI model trained on a diverse dataset of stored ultrasound scans;
a set of assessment facts provided by human reviewers and other automated sources related to the stored ultrasound scans and used as the basis of a review of the ultrasound scan data;
a set of instructions for the AI model detailing the desired format and writing style for the review of the ultrasound scan data created by the AI model, and providing background medical information for reference; and
a user interface allowing a reviewer to select appropriate items based on findings accuracy, scan completeness, and other teaching points.

2. The system of claim 1, wherein the AI model is a large language model trained on a diverse dataset of the stored ultrasound scans.

3. The system of claim 2, wherein the large language model synthesizes the set of assessment facts and instructions to generate the desired written review output.

4. The system of claim 1, wherein the set of assessment facts comprises binary and categorical facts related to accuracy and completeness of at least one ultrasound scan.

5. The system of claim 1, wherein the set of instructions for the AI model comprises at least one example of desired output and detailed information on proper ultrasound procedures.

6. The system of claim 1, wherein the user interface allows the reviewer to select appropriate items based on findings accuracy, scan completeness, and other teaching points in multiple languages.

7. The system of claim 1, further comprising a translator module that generates reviews in multiple languages.

8. A method for generating feedback on at least one ultrasound scan, comprising the steps of:
receiving the at least one ultrasound scan represented by ultrasound scan data from an ultrasound imaging device;
processing the ultrasound scan data using an artificial intelligence (AI) model trained on a diverse dataset of stored ultrasound scan data;
generating a set of assessment facts based on processed ultrasound scan data;
providing the set of assessment facts and a set of instructions to an AI model; and
generating, via the AI model, a written review of the at least one ultrasound scan represented by the ultrasound scan data and the written review is based on the set of assessment facts and instructions.

9. The method of claim 8, wherein the AI model is a large language model trained on a diverse dataset of stored ultrasound scans.

10. The method of claim 9, wherein the large language model synthesizes the set of assessment facts and instructions to generate a written review in multiple languages.

11. The method of claim 8, wherein the set of assessment facts comprises binary and categorical facts related to accuracy and completeness of the ultrasound scan data.

12. The method of claim 8, wherein the set of instructions for the AI model comprises one or more examples of desired output and detailed information on proper ultrasound procedures.

13. The method of claim 8, further comprising:
receiving, on a user interface, a selection of items based on any of accuracy, scan completeness, and other teaching points.

14. The method of claim 8, further comprising a step of delivering the written review of the at least one ultrasound scan to the healthcare practitioner.

15. A computer-readable medium storing instructions that, when executed by a processor, cause a system to:
receive at least one ultrasound scan data;
process the at least one ultrasound scan data using an artificial intelligence AI model trained on a diverse dataset of stored ultrasound scans and associated findings;
generate a set of assessment facts based on processed at least one ultrasound scan data;
provide the set of assessment facts and a set of instructions to an AI model; and
generate a written review of the at least one ultrasound scan data based on the set of assessment facts and the instructions.

16. The computer-readable medium of claim 15, wherein the diverse dataset of stored ultrasound scans and associated findings comprises data from various types of ultrasound machines and different healthcare settings.

17. The computer-readable medium of claim 16, wherein the diverse dataset of stored ultrasound scans further comprises data from stored ultrasound scans selected from the group comprising of 2D, 3D, and Doppler scans.

18. The computer-readable medium of claim 15, wherein the set of assessment facts includes data related to the accuracy of the at least one ultrasound scan data, the completeness of the at least one ultrasound scan data, and educational points derived from the at least one ultrasound scan data.

19. The computer-readable medium of claim 15, wherein the set of instructions for the AI model comprises guidelines for generating the written review in a specific language selected by the user.

20. The computer-readable medium of claim 15, wherein the user interface comprises options for a reviewer to select teaching points based on the specific type of ultrasound scan being reviewed.
